(19) United States
(12) Patent Application Publication
(10) Pub. No.: US 2003/0083501 A1
(43) Pub. Date: May 1, 2003

(54) Process for preparing paroxetine HCl which limits formation of pink colored compounds

(76) Inventors: IIya Avrutov, Bat Hefer (IL); Gideon Pilarsky, Holon (IL)

Correspondence Address: KENYON & KENYON, ONE BROADWAY, NEW YORK, NY, 10004, US

(21) Appl. No.: 10/172,521

(22) Filed: Jun. 14, 2002

Related U.S. Application Data
(60) Provisional application No. 60/298,603, filed on Jun. 14, 2001.
(60) Provisional application No. 60/326,993, filed on Oct. 5, 2001.
(60) Provisional application No. 60/346,048, filed on Jan. 4, 2002.

(51) Int. Cl. C 07D 4 5/02

(52) U.S. Cl. 546/197

(57) ABSTRACT

The present invention provides a process for preparing paroxetine HCl from paroxetine base which provides paroxetine HCl substantially free of pink-colored compounds or an impurity identified by an HPLC RRT of about 1.5. The processes of the present invention utilize a buffer, a molar ratio of HCl to paroxetine base of less than one, and crystallize/recrystallize in the presence of an effective amount of an anti-oxidants. A preferred way to create a buffer is by using ammonium chloride. A preferred anti-oxidant is ascorbic acid.

The present invention also provides for re-crystalizing paroxetine HCl prepared by the above methods or any other methods in the presence of an effective amount of an anti-oxidant such as ascorbic acid. A preferred solvent system for recrystallization is a mixture of acetone and methanol.

Processes of the present invention can combine these various features.

### CROSS-REFERENCE TO RELATED APPLICATIONS

This application claims priority to provisional applications Serial No. 60/298,603, filed Jun. 14, 2001; Serial No. 60/326,993, filed Oct. 5, 2001 and Serial No. 60/346,048, filed Jan. 4, 2002, the contents of which are incorporated herein by reference.

### FIELD OF THE INVENTION

The present invention relates to paroxetine, more particularly, a process for the preparation of paroxetine HCl.

### BACKGROUND OF THE INVENTION

Paroxetine, (−)-trans-3-[(1,3-benzodioxol-5-yloxy)methyl]-4-(4-fluorophenyl) piperidine; (3S, 4R)-3-[5-(1,3-dioxaindanyl)oxymethyl]-4-(p-fluorophenyl)piperidine, is a 5-hydroxytryptamine (5-HT, serotonin) re-uptake inhibitor having the formula:

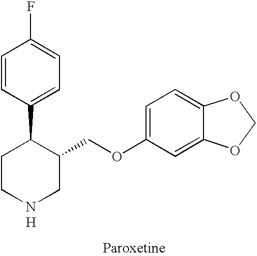

Paroxetine, disclosed in U.S. Pat. No. 4,007,196, is prescribed for the treatment of, inter alia, depression, Parkinson's disease, anxiety disorders, obsessive-compulsive disorders, panic disorder and post-traumatic stress disorder. Other syndromes such as pre-menstrual syndrome (PMS) can also be treated with paroxetine. Paroxetine is marketed as Paxil® in dosage forms containing about 10-40 mg of paroxetine HCl.

A problem with paroxetine HCl tablets is that they often undergo a color change over time. For example, U.S. Pat. No. 6,113,944, discloses that tablets of paroxetine HCl often develop an undesirable pink hue. The '944 patent discloses that formulations of paroxetine HCl prepared in an anhydrous environment have a less likelihood of developing a pink hue.

Without being bound by theory, it is believed that impurities in paroxetine hydrochloride play a role in the color change to pink. The level of the impurities in paroxetine that are associated with a color change to pink can be analyzed in two different manners. One approach is a simple visual analysis, i.e., observing if a sample of paroxetine HCl has turned pink. Another approach is to measure the degree of an impurity identified by a high pressure liquid chromatography (“HPLC”) relative retention time (“RRT”) of about 1.5. The different UV-spectrum characteristic of this impurity has linked the impurity to the development of a pink color. A color change however can occur even if this impurity is present at low levels, suggesting that other impurities may also play a role in the color change. Purification steps to remove this impurity such as by crystallization, extraction, chromatography or other separation procedures are often ineffective.

Thus, there exists a need in the art to prepare paroxetine HCl and its formulations that do not undergo a color change, particularly to pink, during storage.

### SUMMARY OF THE INVENTION

In one aspect, the present invention is directed to a process for preparing paroxetine HCl comprising reacting paroxetine base with less than one base equivalent of HCl, and separating the paroxetine HCl. The molar ratio of HCl to paroxetine base used is preferably from about 0.75 to about 0.95, more preferably from about 0.80 to about 0.90, and most preferably about 0.85

In another aspect, the present invention is directed to a process for preparing paroxetine HCl comprising converting paroxetine base to paroxetine HCl at a pH of greater than about 3.0, and separating the paroxetine HCl. Preferably, the pH is from about 3 to about 8.

In another aspect, the present invention is directed to a process for preparing paroxetine HCl comprising contacting paroxetine base with HCl in a buffer, and separating the paroxetine HCl. Preferably, a weak acidic reagent such as ammonium chloride is added to create a buffer while HCl is added to complete the reaction.

In another aspect, the present invention is directed to a process for preparing paroxetine HCl comprising converting paroxetine base to paroxetine HCl and separating the paroxetine HCl, wherein at least a portion of the process occurrs in the presence of an effective amount of an anti-oxidant and optionally active carbon. A preferred anti-oxidant is ascorbic acid. A preferred amount of ascorbic acid used is from about 0.05 to about 10%, more preferably from about 0.10 to about 10% ascorbic acid (wt/wt % of ascorbic acid to paroxetine base). Preferably, the anti-oxidant is used in combination with active carbon.

In another aspect, the present invention is directed to a process for preparing paroxetine HCl comprising recrystallizing paroxetine HCl in the presence of an effective amount of an anti-oxidant and optionally active carbon, and separating the paroxetine HCl.

The various aspects of the present invention can be combined into a single process. For example, paroxetine base can be contacted with less than one base equivalent of HCl in the presence of a buffer, followed by crystallization in the presence of an anti-oxidant, and optionally active carbon. Alternatively, paroxetine HCl prepared by contacting paroxetine base with less than one base equivalent of HCl and an effective amount of anti-oxidant, can be re-crystallized in the presence of an effective amount of anti-oxidant.

A particularly preferred solvent for the processes of the present invention is toluene, and mixtures of toluene and PGME. A preferred solvent system for re-crystallization of crude paroxetine HCl is a mixture of acetone and methanol.

The present invention is also directed to paroxetine HCl prepared by the processes of and, pharmaceutical compositions thereof containing a pharmaceutically effective amount of paroxetine HCl and a pharmaceutically acceptable excipient, methods of administration thereof.

### FIGURES

FIG. 1 is the HPLC chromatogram for example 2.

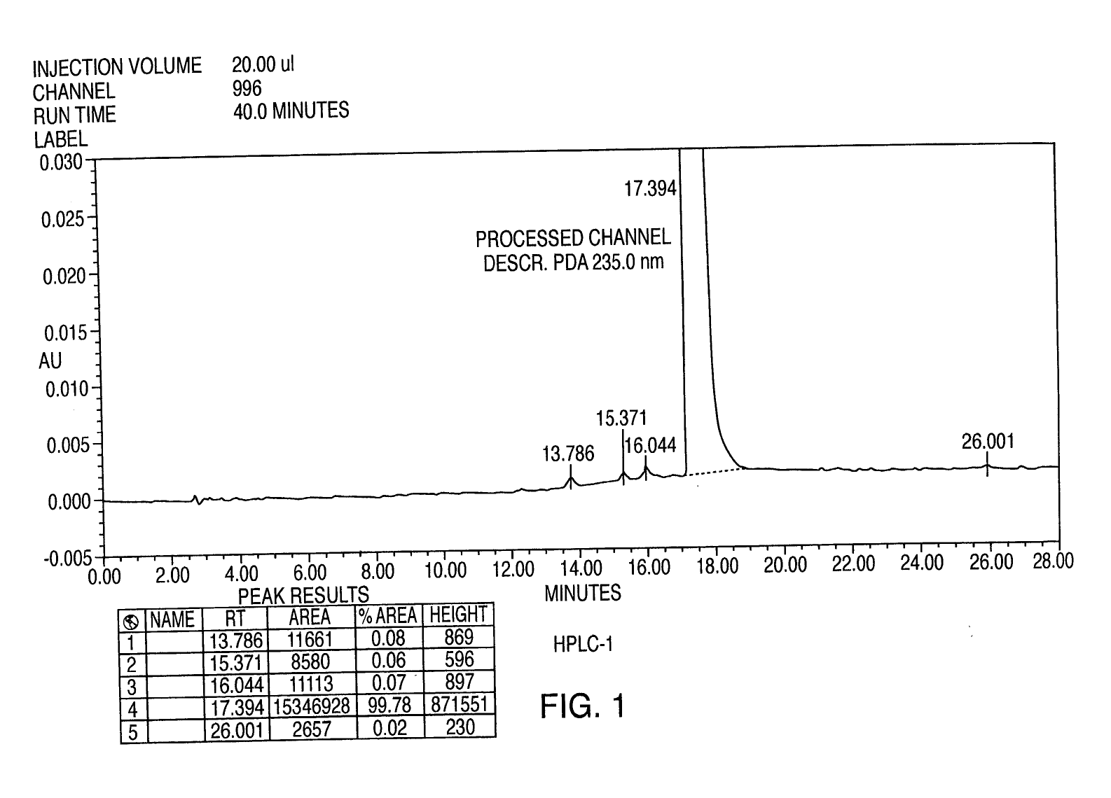

FIG. 2 is the HPLC chromatogram for example 3.

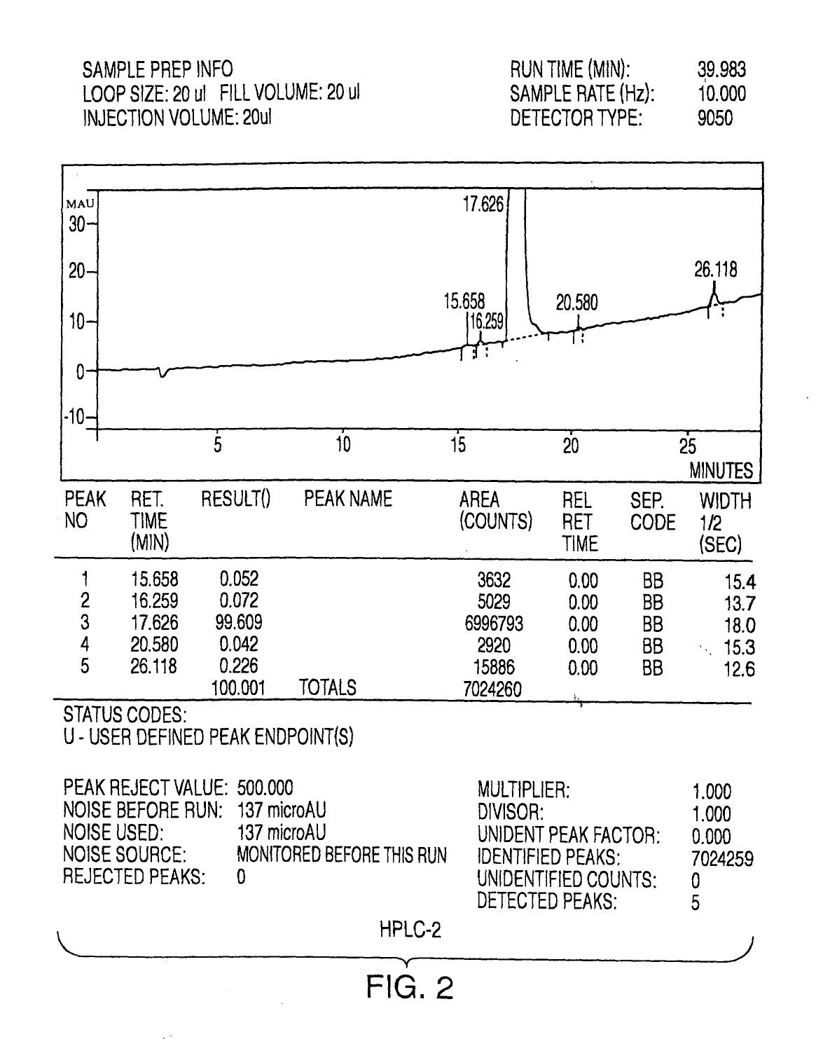

### DETAILED DESCRIPTION OF THE INVENTION

The present invention is directed to novel processes for preparing paroxetine HCl which limit or prevent the formation of pink-colored compounds and/or the amount of an impurity identified by an HPLC RRT of about 1.5 by manipulating the equivalent ratio of HCl, using a buffer, using an anti-oxidant, or a combination thereof. The processes of the present invention limit the formation of impurities believed to be associated with a undesirable color change to pink, including an impurity identified by an HPLC RRT of about 1.5.

As used herein, “pink” has its ordinary meaning and refers to any of a group of colors reddish in hue, of medium to high lightness, and of low to moderate saturation. The term “rose” instead of “pink” is used synonymously in applications to which this application claims priority.

Paroxetine HCl is generally prepared by contacting paroxetine base with a slight excess of concentrated HCl. Such method for conversion however has drawbacks. The use of excess HCl without a buffer can lead to a rapid drop of pH to a pH of about 1 or less. Paroxetine has an acetal group (methylenedioxy), which can hydrolyze relatively easily under such strongly acidic conditions. Additionally, the use of an excess molar ratio of HCl can lead to deterioration of the final product. It is believed that the presence of excess HCl can accelerate acetal hydrolysis by becoming trapped in the final product.

The present invention provides processes designed to address the above drawbacks, thereby limiting the formation of impurities associated with an undesirable change of color to pink.

In one embodiment of the present invention, paroxetine HCl is prepared by contacting paroxetine base with HCl in a buffer. In this embodiment, a weak acid sets up a buffer while HCl is added at an equivalent of less than 1 to complete the conversion to the HCl salt. Preferably, the pH of the reaction mixture is greater than about 3, more preferably from about 3 to about 8.

As used herein, a “weak acid” refers to an acid that does not substantially completely ionize in water. A weak acid has a positive pKa. Ammonium ions, for example, which form as a result of dissociation of ammonium chloride in water, have a pKa of 9.24. An aqueous system employing a weak acid will typically have a pH of above about 3.

The reaction can be carried out by preparing a buffered aqueous solution, and a solution of the base in an organic solvent. The two solutions are then mixed together. Depending on the miscibility of the organic solvent with the aqueous phase, a one or a two phase system is created. Preferably, a one phase system is obtained by using an organic solvent such as toluene that is miscible with the aqueous solution. The mixture of such organic solvents can also be used.

The aqueous solution is buffered by a weak acid. Ammonium chloride is a preferred weak acidic reagent. One of skill in the art can appreciate that ammonium chloride is a salt and its dissolution in an aqueous medium creates ammonium ions, which are the weakly acidic species.

When using a weak acidic reagent such as ammonium chloride, HCl is used to finish the reaction. Particularly when using ammonium chloride, ammonia builds up as the reaction proceeds, resulting in an increase in pH. The addition of HCl maintains a desired pH range.

The organic phase containing paroxetine base can be prepared by dissolving paroxetine base in an organic solvent, or a mixture of such solvents. Examples of such solvents include toluene and glycol monoethers. The use of toluene as a solvent is preferred due to a substantial difference in the solubility of paroxetine base and paroxetine HCl in toluene. Paroxetine base is substantially soluble in toluene, while paroxetine HCl is usually soluble in toluene only at high temperatures, such as reflux. The difference in solubility allows for the crystallization of the HCl salt upon formation thereof, facilitating the separation of the salt and further driving the equilibrium towards salt formation. Other preferred solvents include alcohols such as isopropanol.

Preferably, a mixture of toluene and glycol monoethers is used. The mixture used is preferably from about 8:1 to about 4:1 toluene to glycol monoethers, with a ratio of about 6:1 being preferred. The term “glycol monoethers” refers to the mono-(C₁-C₆, straight- or branched-chain)alkyl ethers of lower alkylene glycols such as, for example, ethylene glycol, propylene glycol, 1,3-butylene glycol and 2,3-butylene glycol. Among preferred glycol monoethers are, for example, ethylene glycol monomethyl ether (“methyl cellosolve”, 2-methoxyethanol), ethylene glycol monoethyl ether (“ethyl cellosolve”, 2-ethoxyethanol) and propylene glycol monomethyl ether (“PGME”, 1-methoxy-2-propanol). Use of PGME is preferred.

After mixing of the two solutions, the base converts to the HCl salt and crystallizes out of the mixture. The resulting mixture can be cooled to accelerate the crystallization of the HCl salt, preferably to a temperature of from about 0° C. to about 10° C., more preferably to below about 5° C. The mixture can also be stirred, both to accelerate conversion to the HCl salt and to induce crystal formation.

The resulting crystals can then be separated by techniques well known in the art, such as filtration. After separation, the crystals can be washed, with an aqueous solvent such as water and a non-aqueous solvent such as toluene and then dried. The product can be dried from a temperature of from about 50° C. to about 80° C. The pressure can be reduced to accelerate the drying process.

In another embodiment, paroxetine base is contacted with less than one base equivalent of HCl in the absence of a buffer. A solution of paroxetine base in an organic solvent or a mixture of solvents such as toluene and monoethers of glycol is prepared as described above. HCl is then added to the solution in a molar ratio of less than one to form paroxetine HCl. Preferably, the molar ratio of HCl to paroxetine base used is from about 0.75 to about 0.95 base equivalent, more preferably from about 0.80 to about 0.90, and most preferably about 0.85.

The solution can be cooled to accelerate the crystallization of the HCl salt, preferably to a temperature of from about 0° C. to about 10° C., more preferably to below about 5° C. The resulting mixture can be stirred, both to accelerate conversion to the HCl salt and to induce crystal formation. If an aqueous medium is used, the pH of the reaction is preferably above about 3, more preferably from about 3 to about 8.

The resulting crystals can then be separated by techniques well known in the art, such as filtration. After separation, the crystals can be washed, with an aqueous solvent such as water and a non-aqueous solvent such as toluene and then dried. The product can be dried from a temperature of from about 50° C. to about 80° C. The pressure can be reduced to accelerate the drying process.

In another embodiment, the HCl salt is prepared by carrying out at least a portion of the preparation of paroxetine HCl in the presence of an anti-oxidant. As used herein, an anti-oxidant has its ordinary meaning in the art and refers to a compound or a chemical substance that inhibits oxidation. One of skill in the art would appreciate that different anti-oxidants known in the art can be used with the present invention. The anti-oxidants used are preferably small organic molecules. Examples of such anti-oxidants include ascorbic acid (Vitamin C), butylated hydroxytoluene (BHT), butylated hydroxyalanine (BHA), with ascorbic acid being preferred. An effective amount of ascorbic acid, preferably from about 0.05 to about 10%, more preferably from about 0.10 to about 10% ascorbic acid (wt/wt % of ascorbic acid to paroxetine base) is used to provide paroxetine HCl product in accordance with the present invention. As one of skill in the art can appreciate, the preferred ratio of other anti-oxidants to paroxetine base can be determined in a routine fashion, with the preferred ratio for ascorbic acid being used as a guidance in such instance.

To crystallize the paroxetine HCl salt, HCl can be added to a solution of paroxetine base and an anti-oxidant in a suitable solvent. In a particularly preferred embodiment, HCl is added at a molar ratio of less than one base equivalent. Preferably, the molar ratio of HCl to paroxetine base used is from about 0.75 to about 0.95 base equivalent, more preferably from about 0.80 to about 0.90, and most preferably about 0.85.

A preferred solvent for the reaction is toluene. Other suitable solvents include alcohols. Preferably, in addition to an anti-oxidant, active carbon is added to the reaction mixture, which further improves decoloration. The amount of active carbon used is preferably from about 0.5 to about 1 gram of active carbon per about 100 ml of solution.

The reaction mixture can be stirred, and the temperature reduced to from about 0° C. to about 10° C., more preferably to below about 5° C., to accelerate crystallization. The formed crystals can then be separated by techniques well known in the art, such as filtration. After separation, the crystals can be washed with toluene and water, and dried to give paroxetine HCl. The product can be dried from a temperature of about 50° C. to about 80° C. The pressure can be reduced to accelerate the drying process. The paroxetine HCl so prepared can optionally be re-crystallyzed in the presence of an effective amount of an anti-oxidant and/or active carbon.

The anti-oxidant can be added at various times during preparation of paroxetine HCl. For example, the anti-oxidant can be present upon contacting paroxetine base with HCl or added after the conversion of the paroxetine base to paroxetine HCl. The presence of the anti-oxidant at least during crystallization of paroxetine HCl is preferred. Preferably, the anti-oxidant is introduced after the conversion to paroxetine HCl, but before crystallization of the HCl salt. In either case, the final product, i.e., paroxetine HCl in solid form, is substantially free of anti-oxidants.

Crytallization in the presence of an anti-oxidant can be used in conjunction with the embodiments in which paroxetine HCl is prepared by using an HCl equivalent of less than one or the embodiment using a buffer, as described herein above. For example, paroxetine base and an effective amount of an anti-oxidant can be dissolved in an organic solvent such as toluene. The resulting solution can then be added to an aqueous solution containing a weak acid. HCl can then be added as described above in a ratio of less than about one base equivalent.

Paroxetine HCl can also be re-crystallized in the presence of an effective amount of an anti-oxidant such as ascorbic acid. To carry out the re-crystallization, paroxetine HCl is dissolved in a suitable organic solvent such as toluene. The toluene is preferably heated to reflux to increase its solubility for paroxetine HCl. Ascorbic acid, preferably with active carbon, is then added to the solution. If active carbon is added, it is subsequently removed, preferably by filtration.

After filtration, the filtrate can be cooled to a temperature of from about 0° C. to about 10° C., with less than about 5° C. being preferred, to accelerate the crystallization process. The crystals are then separated by techniques well known in the art, such as filtration. The crystals can then be washed with an organic solvent such as toluene and a non-organic solvent such as water.

The crude paroxetine HCl prepared by the embodiments of the present invention is preferably recrystallized in an acetone/methanol solvent system, optionally in the presence of an anti-oxidant. Paroxetine HCl is added to a mixture of acetone and methanol, preferably from about a 10:1 to about 30:1, more preferably about a 20:1 mixture. Preferably, an effective amount of ascorbic acid is also added to the mixture. The mixture can be heated, preferably to reflux, to form a solution. The solution is then passed through a charcoal bed to remove impurities. The filtrate is then cooled, preferably to slightly above 0° C., and a precipitate forms. The precipitate, paroxetine hydrochloride hemihydrate, is then separated by techniques well known in the art such as filtration and preferably dried. Two preferred schemes of the present invention are disclosed in Table-1.

TABLE 1
The schemes illustrated are similar,
except scheme II does not use a buffer.

| Preferred Scheme I | Preferred Scheme II |
|---|---|
| <1 molar equivalent of HCl | Same |
| ammonium chloride as a buffer | None |
| Crystallization in the presence of an | Same |
| effective amount of ascorbic acid | |
| Re-crystallization in the presence of an | Same |
| effective amount of ascorbic acid using a | |
| 20:1 mixture of acetone and methanol. | |

The paroxetine hydrochloride of the present process is substantially free of impurities associated with a color change to pink, and is less susceptible, if at all, to develop a pink color overtime These impurities include the impurity identified by an HPLC RRT of about 1.5. Retention time refers to the time required for a compound to pass from the point of injection to the detector. Preferably, the processes of the present invention result in a final product having less than about 0.1% (HPLC area percentage) of the impurity identified by an HPLC RRT of about 1.5. After storage for at least four days at room temperature and a relative humidity of about 60-80%, the level of the impurity identified by an HPLC RRT of about 1.5 is preferably less than about 0.22, more preferably less than about 0.12 and most preferably less than about 0.02 (HPLC area percentage). HPLC area percentage refers to the sum of all the areas under the peak of an impurity in a chromatogram divided by the sum of all the areas under the peaks of all of the other compounds represented in the chromatogram.

The paroxetine hydrochloride of the present invention, in addition to analysis of the amount of the impurity identified by an HPLC RRT of about 1.5, can be analyzed visually for a color change. Preferably, the paroxetine HCl of the present invention remains substantially color-free upon long-term storage. In particular, the paroxetine HCl does not develop a pink color. The paroxetine HCl made in accordance with the present invention can be used to make storage-stable compositions which do not, or are substantially less susceptible, to becoming pink-colored during storage.

One visual analysis can be carried out by preparing a solution of about 2 mg/ml of paroxetine HCl prepared in a mixture of about 0.05M di-Potassium hydrogen phosphate buffer and about 35% of acetonitrile. If the product is substantially free of the impurities associated with a pink color, the solution does not develop a pink color after sitting for about 20 minutes. Preferably, the solution of the paroxetine HCl of the present invention is color free for at least about 20 minutes. On the other hand, available commercial products usually produce a pink colored solution under similar conditions.

Another visual analysis can be carried out by observing the color of paroxetine hydrochloride during storage. Preferably, the paroxetine HCl of the present invention is substantially free compounds associated with a pink color for at least four days at a temperature of about 55° C. and a relative humidity of about 60-80%. One of skill in the art can appreciate that the level of the compounds associated with a pink color can vary according to the temperature and other conditions used for storage.

One of skill in the art can appreciate that the processes of the present invention can be used to prepare different forms of the HCl salt. The HCl salt of paroxetine exists in at least two solid state pseudopolymorph forms differentiated by their degree of hydration. Form I is a non hygroscopic hemihydrate and is thermodynamically more stable. Form II is a hygroscopic anhydrate. Form II converts to Form I if seed crystals of Form I are present, when exposed to humid conditions, or if subject to compression. Commercial paroxetine tablets such as Paxil® usually contain paroxetine HCl hemihydrate.

Paroxetine HCl also exists in other polymorphic forms and solvates of various different solvents. A particularly preferred solvate is the isopropanolate.

The processes of the prior art can be modified according to the teachings of the present invention to prepare the various forms of paroxetine HCl. Crude paroxetine HCl hemihydrate can be formed, for example, from a toluenic solution of paroxetine base by contacting the solution of paroxetine base with aqueous HCl followed by crystallization in an appropriate solvent as generally disclosed in U.S. Pat. No. 4,721,723. Crystalline paroxetine HCl hemihydrate can then be prepared by recrystallization of the crude paroxetine HCl hemihydrate in a suitable solvent. Among suitable solvents are included, for example, lower alkanols such as methanol and ethanol; ketones such as acetone; esters such as ethyl acetate; and, mixtures of any of the foregoing such as methanol/acetone.

The prior art discloses various processes for preparing anhydrous forms of paroxetine HCl, as generally disclosed for example in U.S. Pat. No. 6,080,759. The prior art discloses preparing anhydrous paroxetine HCl by contacting, in a dry N₂ environment, a solution of paroxetine base in an organic solvent, such as isopropanol, with dry HCl gas. Alternatively, the solution of paroxetine base in an organic solvent can be contacted with a solvent substantially free of water wherein the solvent has dry HCl gas dissolved therein. These prior art processes can be modified for crystallization in the presence of ascorbic acid or the use of a certain molar ratio of HCl.

Paroxetine hydrochloride anhydrate can be prepared via the hemihydrate or other solvates. As disclosed in U.S. Pat. No. 6,080,759, anhydrate forms of paroxetine free of bound solvent can also be prepared from the paroxetine hemihydrate by dissolving the hemihydrate in an appropriate solvent substantially free of water which forms an azeotrope with water. Suitably, solvent is removed by distillation and fresh solvent is added until all of the water is removed.

Paroxetine HCl anhydrate can also be made by crystallizing paroxetine HCl in an organic solvent or a mixture of solvents which form a solvate with the paroxetine HCl and displacing the solvated solvent or solvents from the paroxetine HCl solvate using a displacing agent. Preferably, gaseous or liquid water can be used as the displacing agent. It is important that the paroxetine HCl solvate is contacted with enough water and for sufficient time to displace the solvent but insufficient to cause conversion to the HCl hemihydrate.

Paroxetine HCl can also be prepared in various solvate forms as disclosed in U.S. Pat. No. 6,080,759, the processes of which can be modified according to the teachings of the present invention. Among the preferred solvate forms is paroxetine HCl isopropanolate as disclosed for example in Examples 1-3 of U.S. Pat. No. 6,080,759. Paroxetine HCl isopropanolate can be formed by displacing water from paroxetine HCl hemihydrate in, e.g., a mixture of toluene and isopropanol followed by crystallization. Paroxetine HCl isopropanolate can also be formed by contacting a solution of paroxetine base in isopropanol with dry HCl gas followed by crystallization. The isopropanolate can also be formed by contacting a solution of paroxetine base in dry isopropanol with a solution of dry HCl gas in dry isopropanol followed by crystallization. Solvates other than the isopropanolate can be made by similar methods as disclosed in U.S. Pat. No. 6,080,759. Among such solvates are included solvates from solvents such as alcohols other than isopropanol such as 1-propanol and ethanol; from organic acids such as acetic acid; from organic bases such as pyridine; from nitriles such as acetonitrile; from ketones such as acetone and butanone; from ethers such as tetrahydrofuran; from chlorinated hydrocarbons such as chloroform and from hydrocarbons such as toluene. These solvates can be used to form the anhydrate forms free of bound solvent by either displacing the solvent as described above or by removing the solvent by conventional techniques such as vacuum oven drying.

The term paroxetein HCl as used in the present invention includes all these and other polymorphs, solvates and forms of paroxetine hydrochloride.

In accordance with the present invention, the highly pure forms of paroxetine HCl prepared by the new methods disclosed herein can be prepared as pharmaceutical compositions that are particularly useful for inhibiting the re-uptake of serotonin. Such compositions can include any of the various forms of the HCl salt in combination with pharmaceutically acceptable carriers and/or excipients known to one of skill in the art.

For example, these compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with a hydrophilic or a hydrophobic vehicle. For topical administration, suitable transdermal delivery systems known in the art, and for nasal delivery, suitable aerosol delivery systems known in the art, may be employed.

A particularly preferred unit dosage form is a coated tablet. Such tablet contains a pharmaceutically effective amount of the paroxetine HCl of the present invention in conjunction with one or more excipients, such as a binder, filler, stabilizer, disintegrant, glidant, flavoring and coloring agents. An effective amount of paroxetine HCl is approximately from about 10 mg to about 200 mg of the base equivalent of paroxetine HCl, as disclosed in U.S. Pat. No. 6,080,759, more preferably from about 10 mg to about 100 mg, and most preferably from about 10 to about 50 mg.

Suspensions, containing a dosage of about 10 mg of the base equivalent of paroxetine HCl per 5 ml of liquid are also included within the scope of the pharmaceutical compositions of the present invention. The effective dose for the suspension is about the same as that for the tablet.

The prescribing information for Paxil® can be used as a guidance for both dosage and formulation of the paroxetine HCl of the present invention.

Instrumentation Used

HPLC was performed on a XTERRA RP18 (5 um; 250×4.6 mm), reverse phase column with diammonium-hydrogen-phosphate buffer solution: acetonitrile mixture as gradient eluent. Detected by U.V. spectroscopy at λ=285 nm.

### EXAMPLES

### Example 1

Preparation of Paroxetine HCl With a Buffer

An aqueous solution of ammonium chloride (2 grams) in water (5 ml) was added to a solution of paroxetine base (5 grams) in toluene (25 ml). The reaction mixture was intensively stirred at ambient temperature while concentrated HCl was added in such manner that the pH of the reaction mixture stayed between 3.5 and 8. The stirring was continued for 1 hour. A precipitate formed which was filtered and then washed with toluene and water. The resulting material was dried at a temperature of 60° C. under vacuum to give 4.9 grams of paroxetine HCl.

To test the purity of the final product, a 2 mg/ml solution of paroxetine HCl was prepared in a mixture of 0.05M di-Potassium hydrogen phosphate buffer and 35% of acetonitrile. The solution did not develop a pink color after standing for 20 minutes.

### Example 2

Preparation of Paroxetine HCl With a Buffer and an HCl Molar Equivalent of Less Than 1

A solution of ammonium chloride (21.6 grams) in water (80 mL) was added to a solution of paroxetine base (53.2 grams), toluene (480 mL) and propyleneglycol monomethyl ether (PGME) (80 mL). HCl (15.7 grams, 0.85 equivalent, 32%) was then added. The mixture was cooled to 2-3° C., and stirred for 2.5 hours at this temperature (pH of water phase of reaction mixture was 7.5). The formed precipitate was filtered, washed with water and toluene, and dried at a temperature of 60° C. under vacuum to give 48 grams of paroxetine. The content of the impurity at RRT about 1.5 after storage for 4 days at 55° C. was 0.02.

### Example 3

Preparation of Paroxetine HCl Without a Buffer and an HCl Molar Equivalent of About 1

Example 2 was repeated, except the amount of HCl used was 18.5 grams (1 equivalent). The pH of the aqueous phase of the reaction mixture was about 1. The content of the impurity in the product (49.8 grams) after storage for 4 days at 55° C. was 0.23.

### Example 4

Preparation of Paroxetine HCl in the Presence of Ascorbic Acid

Concentrated HCl (2.43 grams) was added to a solution of paroxetine base (5.6 grams) and ascorbic acid (84 mg) in toluene (56 ml). The reaction mixture was stirred at room temperature for 30 minutes, and subsequently cooled to a temperature of 2-4° C. The mixture was kept at this temperature for about 1.5 hour. A precipitate formed. The formed precipitate was filtered, washed with toluene (5 ml) and water (5 ml), and dried at 60° C. under vacuum to give paroxetine HCl of white color (approximately 5 grams).

### Example 5

Recrystallization of Paroxetine HCl in the Presence of Ascorbic Acid and Active Carbon

Paroxetine HCl (approximately 4 grams) was dissolved in toluene(40 ml) at reflux. Ascorbic acid (40 mg) and active carbon SX1 (200 mg) were added to the solution and stirred for 5-10 minutes. The solution was then filtered. The filtrate was cooled to 2-4° C., stirred for approximately 1 hour and filtered again to separate a formed precipitate. The solid precipitate was washed with toluene (4 ml) and dried at a temperature of 60° C. under vacuum to give white (color-free) product (3.4 grams). The product was color-free during storage for at least one month at a temperature of 55° C., and yielded solutions (carried out in the same manner as example 1) that were also color-free.

### Example 6

Preparation of Paroxetine HCl Hemihydrate Crystals

Paroxetine HCl crude (40 g), acetone (400 ml) and methanol (20 ml) and ascorbic acid (0.2 g) are added to a 1L flask. The mixture is heated to reflux, resulting in a solution. The stirring is continued for 15 minutes, after which the hot solution is filtered through a charcoal bed. The filter cake is washed with 5 ml of a mixture acetone/methanol (20:1). The combined filtrates are cooled at 2-3° C. and stirred for 1.5 hours. The precipitate is filtered, washed with acetone (40 ml) and dried to give 35 g of paroxetine HCl hemihydrate crystals.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety.

What is claimed is:

1. A process for preparing paroxetine HCl comprising reacting paroxetine base with less than about one molar base equivalent of HCl and separating the paroxetine HCl, thereby providing a paroxetine HCl substantially free of pink-colored compounds or the amount of an impurity identified by an HPLC RRT of about 1.5.

2. The process of claim 1, wherein the ratio of the HCl to the paroxetine base is from about 0.75 to about 0.95 base equivalent.

3. The process of claim 2, wherein the ratio is from about 0.80 to about 0.90 base equivalent.

4. The process of claim 3, wherein the ratio is about 0.85 base equivalent.

5. The process of claim 1, wherein the reaction has a pH of from about 3 to about 8.

6. The process of claim 5, wherein the reaction takes place in a buffer.

7. The process of claim 6, wherein the buffer is a weak acid created by adding ammonium chloride to an aqueous medium.

8. The process of claim 1, wherein at least a portion of the process is carried out in the presence of an effective amount of an anti-oxidant and optionally active carbon.

9. The process of claim 8, wherein the anti-oxidant is ascorbic acid.

10. The process of claim 1, further comprising re-crystallizing the paroxetine HCl in the presence of an effective amount of an anti-oxidant and optionally active carbon.

11. The process of claim 10, wherein the anti-oxidant is ascorbic acid.

12. The process of claim 1, further comprising recrystallizing the paroxetine HCl from a mixture of methanol and acetone.

13. The process of claim 12, wherein the recrystalization is carried out in the presence of an effective amount of an anti-oxidant and optionally active carbon.

14. The process of claim 13, wherein the anti-oxidant is ascorbic acid.

15. The paroxetine HCl prepared by the process of claim 1.

16. A process of preparing paroxetine HCl comprising contacting paroxetine base with HCl at a pH of from about 3 to about 8, and separating the paroxetine HCl, thereby providing a paroxetine HCl substantially free of pink-colored compounds or the amount of an impurity identified by an HPLC RRT of about 1.5.

17. The process of claim 16, further comprising re-crystallizing the paroxetine HCl in the presence of an effective amount of an anti-oxidant and optionally active carbon.

18. The process of claim 16, further comprising re-crystallizing the paroxetine HCl from a mixture of acetone and methanol.

19. The process of claim 16 or 18, wherein at least a portion of the process is carried out in the presence of an effective amount of an anti-oxidant and optionally active carbon.

20. The process of claim 16, wherein molar ratio of the HCl used is less than about one base equivalent.

21. The paroxetine HCl prepared by the process of claim 16.

22. A process of preparing paroxetine HCl comprising contacting paroxetine base with HCl in a buffer and separating the paroxetine HCl, thereby providing a paroxetine HCl substantially free of pink-colored compounds or the amount of an impurity identified by an HPLC RRT of about 1.5.

23. The process of claim 22, wherein the reaction is buffered with a weak acid.

24. The process of claim 23, wherein the weak acid is a result of addition of ammonium chloride to an aqueous medium.

25. The process of claim 22, wherein the paroxetine base is contacted with less than about 1 molar equivalent of HCl.

25. The paroxetine HCl prepared by the process of claim 22.

26. A process for preparing paroxetine HCl comprising converting paroxetine base to paroxetine HCl, and separating the paroxetine HCl, wherein at least a portion of the process is carried out in the presence of an effective amount of an anti-oxidant, thereby providing a paroxetine HCl substantially free of pink-colored compounds or the amount of an impurity identified by an HPLC RRT of about 1.5.

27. The process of claim 26, wherein the anti-oxidant is selected from the group consisting of ascorbic acid, BHT and BHA.

28. The process of claim 27, wherein the amount of ascorbic acid used is from about 0.05% to about 10% weight of paroxetine HCl.

29. The process of claim 28, wherein the ascorbic acid is from about 0.1% to about 10% weight of paroxetine HCl.

30. The process of claim 26, wherein paroxetine base is converted to paroxetine HCl by contacting paroxetine base with less than about one base equivalent of HCl.

31. The process of claim 30, wherein the conversion takes place from a pH of from about 3 to about 8.

32. The process of claim 31, wherein the pH is buffered.

33. The process of claim 26, further comprising recrystallizing the paroxetine HCl in the presence of an effective amount of an anti-oxidant.

34. The process of claim 26, further comprising recrystallizing paroxetine HCl from a mixture of methanol and acetone.

35. The process of claim 34, wherein the re-crystallization is carried out in the presence of an effective amount of an anti-oxidant.

36. The paroxetine HCl prepared by the process of claim 26.

37. A process for preparing paroxetine HCl comprising the steps of:
a) reacting paroxetine base with less than about 1 molar equivalent of HCl in the presence of ammonium ions;
b) crystallizing the paroxetine HCl in the presence of an effective amount of an anti-oxidant and optionally active carbon;
c) separating the paroxetine HCl; and
d) re-crystallizing the paroxetine HCl, optionally in the presence of an anti-oxidant.

38. The process of claim 37, wherein the re-crystallization is carried out from a mixture of acetone and methanol.

39. The process of claim 37, wherein the anti-oxidant is ascorbic acid.

40. A process for preparing paroxetine HCl comprising the steps of:
a) reacting paroxetine base with less than about 1 molar equivalent of HCl;
b) crystallizing the paroxetine HCl in the presence of an effective amount of an anti-oxidant and optionally active carbon;
c) separating the paroxetine HCl; and
d) re-crystallizing the paroxetine HCl, optionally in the presence of an anti-oxidant.

41. The process of claim 40, wherein the re-crystallization is carried out from a mixture of acetone and methanol.

42. The process of claim 40, wherein the anti-oxidant is ascorbic acid.

43. Paroxetine HCl characterized by a having about 0.1% or less of an impurity identified by an HPLC RRT of about 1.5.

44. Paroxetine HCl characterized by less than about 0.22 of an impurity identified by an HPLC RRT of about 1.5 after storage for at least four days at a temperature of about 55° C., and that upon visual inspection does not appear pink.

45. The paroxetine HCl of claim 44, wherein the impurity is less than about 0.12

46. The paroxetine HCl of claim 45, wherein the impurity is less than about 0.02.

47. The paroxetine HCl of claim 43 or 44, wherein the paroxetine HCl does not appear pink upon visual inspection.

48. The paroxetine HCl of claim 43 or 44 wherein the paroxetine HCl is paroxetine HCl hemihydrate.

49. The paroxetine HCl of claim 43 or 44, wherein the paroxetine HCl is paroxetine HCl anhydrate.

50. The paroxetine HCl of claim 43 or 44, wherein the paroxetine HCl is a solvate of a solvent selected from the group consisting of isopropanol, 1-propanol, ethanol, acetic acid, pyridine, acetonitrile, acetone, butanone, tetrahydrofuran and toluene.

51. A pharmaceutical composition of paroxetine HCl comprising an effective amount of paroxetine HCl of claim 43 or 44, and a pharmaceutically acceptable excipient.

52. A method for inhibiting the re-uptake of serotonin in a mammal in need thereof comprising administering the pharmaceutical composition of claim 51.

53. A method for treating a disease or syndrome selected from the group consisting of depression, Parkinson's disease, anxiety disorders, obsessive-compulsive disorders, panic disorder, post-traumatic stress disorder and PMS comprising administering the pharmaceutical composition of claim 51.
